(19) United States
(12) Patent Application Publication
(10) Pub. No.: US 2007/0196278 A1
(43) Pub. Date: Aug. 23, 2007

(54) Compositions and methods for locating an internal bleeding site

(75) Inventors: Seth Karp, Chestnut Hill, MA (US); Evan Garfein, Boston, MA (US)

Correspondence Address: Medlen & Carroll, LLP, Suite 350, 101 Howard Street, San Francisco, CA, 94105, US

(73) Assignee: Cold Spring Diagnostics, Inc., Chestnut Hill, MA (US)

(21) Appl. No.: 11/633,193

(22) Filed: Dec. 4, 2006

Related U.S. Application Data
(63) Continuation-in-part of application No. 10/903,303, filed on Jul. 29, 2004.

(51) Int. Cl. A61K 51/00; A61K 49/00

(52) U.S. Cl. 424/1.69; 424/9.6

(57) ABSTRACT

The present invention relates to medical diagnostic techniques and more particularly to compositions and methods for locating an internal bleeding site in a subject.

This application is a continuation-in-part of U.S. patent application Ser. No. 10/903,303, filed Jul. 29, 2004, the disclosure of which is herein incorporated by reference in its entirety.

### FIELD OF THE INVENTION

The present invention relates to medical diagnostic techniques and more particularly to compositions and methods for locating an internal bleeding site in a subject.

### BACKGROUND OF THE INVENTION

Bleeding is a common reason for hospitalization and outpatient treatment, particularly in older individuals. Disease or trauma can cause a hemorrhage at virtually any location in the body. Bleeding through the skin is easily identified, since it is clearly visible. However, bleeding within an internal cavity or organ of the body can be much more difficult to identity. As such, critical treatment may be delayed as time is spent attempting to localize the internal hemorrhage.

Internal bleeding is an important health problem. Approximately 1 in 5 people will experience at least one episode of significant internal bleeding during their lifetime. About half of these episodes are due to bleeding from the colon. The most common cause of colonic bleeding is diverticulosis. Approximately sixty-five percent (65%) of people develop this condition by age eighty-five. Fifteen percent (15%) of these people, or approximately ten percent (10%) of the entire population will experience significant bleeding as a result.

Many diagnostic techniques now exist to localize an area of internal hemorrhage. These techniques include endoscopy, angiography and nuclear medicine scans.

Endoscopy involves the placing of an optical scope into a body orifice such as the esophagus, stomach, or large bowel. Once a bleeding site is visualized, treatment is often possible using well-known techniques such as cauterization or banding. In general, this technique requires that active bleeding occur during the viewing procedure. Bleeding sites may be difficult to identify because of obscuration by blood and the fact that certain regions of the bowel and most internal organs are inaccessible to scopes.

Angiography is an invasive procedure involving the passage of a catheter into the patient's aorta through an entry site, usually in the leg. Dye is injected from the end of the catheter when the catheter is located adjacent to vessels in which bleeding is likely occurring. The dye pools in an area of active bleeding, producing a characteristic blush that can be seen using an X-ray camera. There are many disadvantages of this technique. The dye can cause reactions within the body resulting in kidney failure or even death. Serious bleeding can occur at the site through which the catheter is inserted, and at times this requires an operation to repair. In addition, high doses of X-rays are required to perform this test.

Finally, a nuclear medicine scan involves the injection, into the patient's blood stream, of a radioactive marker that attaches to red blood cells. The cells are traced to an active bleeding site using remote scanners sensitive to radiation.

Each of the diagnostic tests described above requires active bleeding to reveal the presence of an internal hemorrhage. It is often the case, however, that by the time the diagnostic tests are performed, a clot produced by the body has stopped the bleeding, so the test does not provide useful information. Without definitive treatment of the bleeding source about half of patients will experience a recurrence of the hemorrhage. This is a dangerous situation because the bleeding may occur at any time, even after the patient has left the hospital.

The ability to localize a bleeding source, even when no active bleeding is occurring, would allow definitive treatment in many cases and greatly reduce the potential harm caused by bleeding. In cases of particularly severe hemorrhage, the inability to accurately localize a bleeding site may mean that the surgery required to correct it ends up being much larger in scale than would be required if the bleeding source had been clearly identified. The disadvantages to a larger operation are clear. It increases time and costs, increases complications and requires a longer recovery time. For example, if a patient has a life-threatening bleeding condition in the colon, inability to accurately define the region necessitates removal of the entire colon. Conversely, if localization were possible, the patient might lose only one quarter of the entire colon. The latter operation is shorter, simpler and the patient suffers no substantial disability following the operation.

### SUMMARY OF THE INVENTION

The present invention provides compositions and methods for localizing an internal bleeding site whereby a protein or other factor involved in the clotting process is complexed to an imaging agent and injected into a patient believed to be at risk of internal bleeding. A clot in the patient will naturally accumulate a certain concentration of the injected complex, and within a short period of time, the concentration becomes sufficient to be detected by an imaging apparatus. The imaging contrast agent may, for example, be an MRI contrast agent, a CT contrast agent, a PET agent, a fluorescent substance, a luminescent substance, and the like.

In some embodiments, the present invention provides compositions and method for localizing an internal bleeding site/hemorrhage that does not require substantial and invasive procedures or internal visible observations. The invention operates by recognizing a consequence of the bleeding—i.e., a clot formed by the body at the site of bleeding. This clot is produced during active bleeding and persists after the bleeding is stopped, so that the invention enables a bleeding site to be located regardless of whether the site is bleeding actively or is clotted. The invention, moreover, enables localization with a high degree of accuracy under a variety of conditions.

For example, in some embodiments, the present invention provides methods and compositions (e.g., kits, mixtures, pharmaceutical preparations, systems) for localizing an internal bleeding site in the body of an animal (e.g., a mammal, a human), comprising: introducing an optically labeled targeting component into the animal and detecting an internal bleeding site in the animal by detecting localization of the optically labeled targeting component. In some embodiments, the targeting component is fibrinogen. In some embodiments, the targeting component is optically labeled platelets, Factors I, Ia, II, IIa, V, Va, VII, VIIa, VIII, VIIIa, IX, IXa, X, Xa, XI, XIa, XII, XIIa, XIII, XIIIa, fibrinogen, fibrin, fibronectin, von Willebrand's Factor, vinculin, vitronectin, Factor VIIIa and/or b component peptides, ADP, serotonin, platelet factor 4, bethathromboglobulin, high-molecular-weight kininogen, kallikrein, prekallikrein, antithrombin II, protein C, talin, or fibrin-stabilizing factor. In some embodiments the optical label is a fluorophore.

In some embodiments, the present invention provides a method for localizing an internal bleeding site in the body of a mammal believed to be at risk of internal bleeding, the method comprising: introducing into the circulatory system of the mammal a solution comprising a clotting factor that is capable of contributing to clot formation, and a contrast agent complexed to the clotting factor; permitting time to pass for at least some of the clotting factor in the solution to become localized to the bleeding site to participate in clot formation; scanning the body of the mammal near a suspected bleeding site with a detector capable of detecting the contrast agent so as to determine a location of the bleeding site based upon a concentration of the contrast agent complexed to the clotting factor. In some embodiments, the contrast agent comprises at least one of an MRI contrast agent, a CT contrast agent, a PET agent, and a fluorescent substance. In some embodiments, the MRI contrast agent comprises at least one of a paramagnetic contrast agent, a ferromagnetic contrast agent, and a superparamagnetic contrast agent. In some embodiments, the MRI contrast agent comprises at least one of: titanium, vanadium, chromium, manganese, iron, cobalt, nickel, copper, europium, gadolinium, protactinium, magnetite and gamma ferric oxide. In some embodiments, the CT contrast agent comprises at least one of iron, calcium, barium and iodine. In some embodiments, the PET contrast agent comprises Fluorine 18. In some embodiments, the fluorescent substance comprises indocyanine green, iodocyanine green, IRDye78, IRDye80, IRDye38, IRDye40, IRDye41, IRDye700, IRDye800, Cy7, IR-786, DRAQ5NO, quantum dots, and analogs thereof. In some embodiments, the clotting factor comprises any protein shown to localize to an area of a clot. In some embodiments, the contrast agent comprises an MRI contrast agent, and the body of the mammal is scanned by placing the mammal in an MRI scanner so as to determine the location of the bleeding site. In some embodiments, the contrast agent comprises a CT contrast agent, and the body of the mammal is scanned by placing the mammal in a CT scanner so as to determine the location of the bleeding site. In some embodiments, the contrast agent comprises a PET agent, and the body of the mammal is scanned by placing the mammal in a PET scanner so as to determine the location of the bleeding site. In some embodiments, the contrast agent comprises a fluorescent substance, and the mammal is scanned by placing a scope into a body cavity suspected of bleeding and exciting and detecting a fluorescent substance so as to determine the location of the bleeding site. In some embodiments, the contrast agent comprises a fluorescent substance, and the mammal is scanned while in the operating room with an open body cavity by placing suitable camera over the area of the suspected bleeding site and detecting a fluorescent substance so as to determine the location of the bleeding site.

The present invention is not limited by the site of the bleeding detection. In some embodiments, the bleeding site is located in the colon. In some embodiments, the bleeding site is located in the small intestine. In some embodiments, the bleeding site is located in the stomach. In some embodiments, the bleeding site is located in the esophagus.

The present invention also provides composition and methods for detecting bleeding by using a combination of: a) an optically labeled targeting component configured to accumulate in a clot; and b) a radioactively labeled targeting component configured to accumulate in a clot. In some embodiments, the present invention provides kits comprising the two components. In some embodiments, the kits comprise: a) an optically labeled targeting component configured to accumulate in a clot; b) an unlabeled targeting component and reagents for radioactively labeling said unlabeled targeting component. In some embodiments, the compositions and methods employ components provided in a pharmaceutically acceptable solution. In some embodiments, the compositions and methods employ the components in pharmaceutical dosage form for administration to a subject at 1 to 100 microcuries/kg of body weight of the subject of radioactively labeled targeting component and 100 micrograms to 100 milligrams/kg of body weight of the subject of optically labeled targeting component.

U.S. Pat. No. 6,314,314 issued to the inventor of the present invention discloses a technique for localizing an internal bleeding site using a radioactively labeled clotting factor, but the method disclosed in U.S. Pat. No. 6,314,314 is not applicable to MRI, CT, X-ray, PET, or optical imaging techniques.

### BRIEF DESCRIPTION OF THE DRAWINGS

FIG. 1 shows a schematic diagram of an exemplary bleeding site within a human colon.

FIG. 2 shows a schematic diagram showing the administration of the diagnostic procedure according to an embodiment of the present invention.

FIG. 3 shows a flow diagram showing the blood coagulation cascade according to a first known model.

FIG. 4 shows a flow diagram showing the blood coagulation cascade according to a second known model.

FIG. 5 shows synthesis and optical properties of FBG800. A, The labeling ratio of FBG800 as a function of the protein to fluorophore mixing ratio. B, Relative per fluorophore fluorescence, as a function of labeling ratio, compared to free CW800-CA (1 μmol/L) in PBS. C, Per molecule total fluorescence yield calculated from the labeling ratio and per fluorophore fluorescence, and compared to the same dye concentration (1 μmol/L) of free CW800-CA in PBS (closed circles). The relative ratio of bioactivity of FBG800 to native FBG measured using a fibrin polymerization assay (open circles). D, Absorbance spectra of CW800-CA and FBG800 (labeling ratio=1.2) in PBS. Peak absorption of CW800-CA and FBG800 was 775 nm and 785 nm, respectively. E, Emission spectra of CW800-CA and FBG800 (labeling ratio=1.2). Peak emission of CW800-CA and FBG800 was 794 nm and 800 nm, respectively. F, The conjugation sites of CW800 to FBG (white) were established using trypsin digestion and electrospray time-of-flight liquid chromatography/mass spectrometry peptide analysis, and are shown overlaid with the crystal structure¹⁸ of the dimer. The amino acid substitution sites are: Lys399 (upper circle on left side; lower circle on right side) and Lys185 (lower circle on right side; upper circle on left side) of the Bβ chain. FBG, Fibrinogen.

FIG. 6 shows real-time intraoperative thrombus detection using invisible light. A, FBG800 (left) or HSA800 (right) was injected into the mouse 30 minutes after injury of a mesenteric vessel (arrows). Shown are color video (left of each set) and NIR fluorescence (right of each set). All NIR fluorescence images have identical exposure times and normalizations. Results are representative of N=4 animals. B, FBG800 was injected into the pig 30 minutes after injury (arrows) of a mesenteric vessel (top) or mucosal resection of the stomach (bottom). Shown are color video (left), NIR fluorescence (right), and a pseudocolored (yellow/green) merge of the two (right). Results are representative of N=3 animals. FBG, Fibrinogen; HSA, human serum albumin; NIR, near-infrared.

### DETAILED DESCRIPTION OF THE INVENTION

Certain illustrative embodiments of the present invention are described below. The present invention is not limited to these particular embodiments.

Imaging Agents

The present invention contemplates the use of a wide variety of imaging agents in the compositions and methods of the present invention. The imaging contrast agent may, for example, be an MRI contrast agent, a CT contrast agent, a PET agent, a fluorescent substance, a luminescent substance, and the like.

The term “contrast agent” comprises those agents that enhance image contrast during diagnostic imaging. In a preferred embodiment, a contrast agent is one that enhances contrast in MRI or optical imaging.

The most frequently used intravenous contrast agents are based on chelates of gadolinium. Contrast agents include, but are not limited to: MAGNEVIST (Schering AG), PROHANCE (Bracco Pharmaceuticals, Inc.), Gadomer-17 (Schering AG), B22956/1 (Bracco Pharmaceuticals, Inc.) and the contrast agents disclosed in U.S. Pat. No. 5,649,537, MP-2269 (Mallinckrodt, Inc.) and the contrast agents disclosed in international patent application (PCT) WO 98/20908, P760 and P775 (Guerbet SA), CLARISCAN (Nycomed Amersham), COMBIDEX and FERRIDEX (Advanced Magnetics, Inc.), gadolinium texaphrin (Pharmacyclics, Inc.), and Eovist (Schering AG) for MRI; and iohexol, iopamidol, iopromide, iopentol, and ioxaglate for CT. In some embodiments, the CT contrast agent is any radio-opaque substance that can be complexed with a targeting component of the invention (e.g., any radio-opaque substance complexed with fibrinogen).

In some embodiments, fluorescent agents are used as the imaging agent. Generally preferred fluorophores are those which efficiently fluoresce upon excitation with light whose wavelength falls within the range of about 300 to about 800 nanometers, preferably in the range of about 500 to 800 nanometers. Suitable fluorophores include those that absorb and/or emit at wavelengths that are distinguishable from the excitation and emission maxima of the other components in the environment to minimize background fluorescence. For those methods involving detection or imaging during surgery, fluorophores having excitation and/or emission wavelengths of at least about 500 nanometers are preferred. The use of such fluorophores reduces interference from the ambient fluorescence of other biological components. Preferred fluorophores also exhibit a high degree of fluorescence polarization, preferably greater than about 10% of the theoretical maximum value for an observable polarization.

Preferred fluorophore moieties include fluorescent dyes having (a) high fluorescence intensity, (b) sufficiently long excitation and emission wavelength maxima so that interference from natural fluorescence of injured, diseased, or normal tissue is minimized; (c) sufficiently long measured fluorescence decay time to allow accurate measurement of emitted light over background fluorescence and scattering (at least about 2, preferably at least about 10 nanoseconds); and (d) high degree of fluorescence polarization.

Exemplary fluorophores include, but are not limited to, 5-(and 6) carboxynaphtofluorescein, Texas Red, BODIPY 630/650, which can be conveniently coupled to primary amines using an active ester derivative that is available commercially (Molecular Probes, Inc., Catalog number, B-10003), BODIPY 650/665, which can be conveniently coupled to primary amines using an active ester derivative that is available commercially (Molecular Probes, Inc., Catalog number, B-10005), Dansyl, which can be conveniently coupled to primary amines using dansyl chloride that is commercially available (Aldrich Chemical), Rhodamine, which can be conveniently coupled to primary amines using an active ester derivative that is available commercially (Molecular Probes, Inc., Catalog number, R-6107), 5-TAMRA, which can be conveniently coupled to primary amines using an active ester derivative that is available commercially (Molecular Probes, Inc., Catalog number, C-2211), coumarin dyes, nitrobenzoxazole dyes, cyanine dyes, dipyrrometheneboron dyes, xanthene dyes (including the benzo- and naphtho-xanthene dyes), phenoxazine dyes (as well as the benzo- and naphtho-phenoxazine dyes) and compounds from other classes of dyes well known to those of skill in the art, 1,5 IAEDANS, 1,8-ANS, 4-Methylumbelliferone, 5-carboxy-2,7-dichlorofluorescein, 5-Carboxyfluorescein (5-FAM), 5-Carboxynapthofluorescein, 5-FAM (5-Carboxyfluorescein), 5-HAT (Hydroxy Tryptamine), 5-Hydroxy Tryptamine (HAT), 5-ROX (carboxy-X-rhodamine), 6-Carboxyrhodamine 6G, 6-CR 6G, 6-JOE, 7-Amino-4-methylcoumarin, 7-Amninoactinomycin D (7-AAD), 7-Hydroxy-4-methylcoumarin, 9-Amino-6-chloro-2-methoxyacridine, ABQ, Acid Fuchsin, ACMA (9-Amino-6-chloro-2-methoxyacridine), Acridine Orange, Acridine Red, Acridine Yellow, Acriflavin, Acriflavin Feulgen SITSA, Aequorin (Photoprotein), AutoFluorescent Protein—(Quantum Biotechnologies) see sgGFP, sgBFP, Alexa Fluor (various), Alizarin Complexon, Alizarin Red, Allophycocyanin (APC), AMC, AMCA-S, AMCA Aminomethylcoumarin), AMCA-X, Aminoactinomycin D, Aminocoumarin, Aminomethylcoumarin (AMCA), Anilin Blue 600, Anthrocyl stearate, APC (Allophycocyanin), APC-Cy7, APTRA-BTC=Ratio Dye, Zn2+, APTS, Astrazon Brilliant Red 4G, Orange R, Red 6B, and Yellow 7 GLL, Atabrine, ATTO-TAG CBQCA and FQ, Auramine, Aurophosphine, BAO 9 (Bisaminophenyloxadiazole), BCECF (high or low pH), Berberine Sulphate, Beta Lactamase, BFP blue shifted GFP, Blue Fluorescent Protein, blue shifted GFP (Y66H), BFP/GFP FRET, Bimane, Bisbenzamide, bis-BTC=Ratio Dye, Zn2+, Blancophor FFG and SV, BOBO-1 and -3, Bodipy (various), BO-PROTM-1 and -3, Brilliant Sulphoflavin, BTC—Ratio Dye Ca2+, BTC-5N, Calcein, Calcein Blue, Calcium Crimson, Calcium Green (various) and Orange, Calcofluor White, Carboxy-X-rhodamine (5-ROX), Cascade Blues, Cascade Yellow, Catecholamine, CCF2 (GeneBlazer), CFDA, CFP=13 Cyan Fluorescent Protein, Cyan Fluorescent Protein, CFP/YFP FRET, Chlorophyll, Chromomycin A, CL-NERF (Ratio Dye, pH), CMFDA, Coelenterazine Dye (various), Coumarin Phalloidin, C-phycocyanine, Methylcoumarin, Methylcoumarin CTC, CTC Formazan, Cy2 and various others, Cyan GFP, cyclic AMP Fluorosensor (FiCRbR), Dabeyl (various), Dansyl (various), DAPI, Dapoxyl (various), DCFDA, DCFH (Dichlorodihydrofluorescein Diacetate), DDAO, DHR (Dihydorhodamine 123), Di-4-ANEPPS, Di-8-ANEPPS, DiA (4Di-16-ASP), Dichlorodihydrofluorescein Diacetate (DCFH), DiD—Lipophilic Tracer, DiD (DiIC18(5)), DIDS, Dihydorhodamine 123 (DHR), DiI (IilC18(3)), Dinitrophenol, DiO (DiOC18(3)), DiR (various), DM-NERF (high pH), DNP, Dopamine, DsRed, DTAF, DY-630-NHS, DY-635-NHS, EBFP, Enhanced Blue Fluorescent Protein, Enhanced Cyan Fluorescent Protein, Enhanced Green-Fluorescent Protein, ELF, Eosin, Erytbrosin, Erythrosin, Ethidium Bromide, Ethidium homodiiner-1 (EthD-1), Euchrysin, EukoLight, Europium (II) chloride, EYFP, Enhanced Yellow Fluorescent Protein, Fast Blue, FDA, Feulgen (Pararosaniline), FIF (Formaldehyd Induced Fluorescence), Flazo Orange, Fluo-3, Fluo-4, Fluorescein (FITC), Fluorescein Diacetate, Fluoro-Emerald, Fluoro-Gold (Hydroxystilbamidine), Fluor-Ruby, FluorX, FM 1-43, FM 4-46, Fura Red (various), Genacryl Brilliant Red B, Brilliant Yellow 10GF, Pink 3G, and Yellow 5GF, GeneBlazer (CCF2), GFP (various), Gloxalic Acid, Granular Blue, Haematoporphyrin, Hoechst 33258, 33342, and 34580, HPTS, Hydroxycoumarin, Hydroxystilbamidine (FluoroGold), Hydroxytryptamine, Indo-1 (various), Indodicarbocyanine (DiD), Indotricarbocyanine (DiR), Intrawhite Cf, JC-1, JO-JO-1, JO-PRO-1, LaserPro, Laurodan, LDS 751, Leucophor PAF, SF, and WS, Lissamine Rhodarnine (various), Calcein/Ethidium homodimer, LOLO-1, LO-PRO-1, Lucifer Yellow, Lyso Tracker (various), LysoSensor (various), Mag Green, Magdala Red (Phloxin B), Mag-Fura Red, Mag-Fura-2, Mag-Fura-5, Mag-Indo-1, Magnesium Green and Orange, Malachite Green, Marina Blue, Maxilon Brilliant Flavin, Maxilon Brilliant Flavin, Merocyanin, Methoxycoumarin, Mitotracker Green, Orange, and Red, Mitramycin, Monobromobimane, Monobromobirmane (mBBr-GSH), GSH), Monochlorobimane, MPS (Methyl Green Pyronine Stilbene), NBD, NBD Amine, Nile Red, Nitrobenzoxadidole, Noradrenaline, Nuclear Fast Red, Nuclear Yellow, Nylosan Brilliant lavin, Oregon Green (various), Pacific Blue, Pararosaniline (Feulgen), PBFI, PE-Cy5 and Cy7, PerCP, PE-TexasRed [Red 613], Phloxin B (Magdala Red), Phorwite (various), Phosphine, PhotoResist, Phycoerythrin B and R, PKH26 and 67, PMIA, Pontochrome Blue Black, POPO-1 and -3, PO-PRO-1 and -3, Primuline, Procion Yellow, Propidium lodid (PI), PyMPO, Pyrene, Pyronine, Pyronine B, Pyrozal Brilliant Flavin, QSY, Quinacrine Mustard, Red 613 [PE-TexasRed], Resorufin, Rhodamine (various), Rose Bengal, R-phycocyanine, R-phycoerythrin (PE), red shifted GFP (various), Sapphire GFP, SBFI, Serotonin, Sevron Brilliant Red, Orange, and Yellow L, SuperGloGFP (various), SITS (various), SNAFL (various), SNARF, Sodium Green, Spectrm qua, Green, Orange, and Red, SPQ (6-methoxy-N-(3-sulfopropyl)quiolinium), Stilbene, Sulphorhodamine B and G, SYTO (various), SYTOX Blue, Green, and Orange, Tetracycline, Tetramethylrhodanine (TRITC), Texas Red, Texas Red-X conjugate, Thiadicarbocyanine (DiSC3), Thiazine Red R, Thiazole Orange, Thioflavin 5, S, and TCN, Thiolyte, Thiozole Orange, Tinopol CBS (Calcofluor White), TMR, TO-PRO-1, -3 and -5, TOTO-1 and -3, TriColor (PE-CyS), TRITC, TetramethylRodamineIsoThioCyanate, True Blue, TruRed, Ultralite, Uranine B, Uvitex SFC, X-Rhodamine, XRITC, Xylene Orange, Y66F, H, and W, Yellow shifted Green Fluorescent Protein, Yellow Fluorescent Protein, YO-PRO-1 and -3, and, YOYO-1 and -3.

In some embodiments, a combination of different imaging agents is used. For example, in some embodiments, an optical imaging agent is used in conjunction with a radioactive imaging agent. In such embodiments, the different imaging agents may be associated with the same or different targeting components.

Targeting Components

The imaging components are associated with targeting components that are clotting components including, but not limited to, platelets, Factors I, Ia, II, Ia, V, Va, VII, VIIa, VIII, VIIIa, IX, IXa, X, Xa, XI, XIa, XII, XIIa, XIII, XIIIa, fibrinogen, fibrin, fibronectin, von Willebrand's Factor, vinculin, vitronectin, Factor VIIIa and/or b component peptides, ADP, serotonin, platelet factor 4, bethathromboglobulin, high-molecular-weight kininogen, kallikrein, prekallikrein, antithrombin II, protein C, talin, and fibrin-stabilizing factor.

The targeting components may be in the form of native molecules or may be fragments, chimeras, or variants thereof, so long as they retain the ability to associate with the clot.

Certain preferred embodiments use fibrinogen (Weisel, Fibrinogen and fibrin, Adv. Protein Chem., 70:247 (2005), herein incorporated by reference in its entirety) or variants thereof.

The imaging components may be associated with the targeting components via any desired mechanism. In some embodiments, the imaging agents are directly or indirectly (e.g., via a linker) covalently conjugated to the targeting component. One or more of the imaging components may be associated with individual targeting components. In preferred embodiments, the imaging component is attached to the targeting component in a location that does not substantially hinder the association of the targeting component to a clot and that does not substantially prevent detection of the imaging component.

In some embodiments, methods of the invention employ existing labeled products alone or in conjunction with novel components described herein for one or more medical uses. Existing labeled products include, but are not limited to, HEPATOLITE (^{99m}Tc-DISIDA) (CIS-US), CARDIOLITE (DuPont), MIRALUMA (DuPont), NEUROLITE (Bristol Meyers Squibb), PULMOLITE (CIS-US), and CIS-PYRO (CIS-US).

Compositions, Kits, and Systems

The present invention provides compositions, kits, and systems that comprise one or more components of the present invention. For example, the present invention provides diagnostic kits for use by researchers or physicians. In some embodiments, the kit comprises labeled targeting components in a pharmaceutically suitable form. For example, in some embodiments, the present invention provides kits comprising fluorescently labeled fibrinogen. In some embodiments, the kits further comprise a radioactively labeled targeting component, which may also be, for example, fibrinogen. In some embodiments, the kit provides unlabeled targeting components and reagents for labeling the targeting component (e.g., reagents to label fibrinogen in a generator). The kit may further comprise control reagents, such as unlabeled targeting component.

In some embodiments, kits comprise components in lyophilized or freeze-dried form. In some embodiments, the kits further comprise a buffer for reconstituting the components in solvated or suspension form for administration to a subject. In some embodiments, the kits comprise components that are in injectable liquid form. In some embodiments, the concentrations of the components are selected for readily providing pharmaceutically acceptable dosing to a subject (e.g., for administration of 1 to 100 microcuries/kg of radiolabeled fibrinogen and/or 100 micrograms to 100 milligrams/kg of fibrinogen complexed to a fluorophore to a subject). In some embodiments, the kits comprise instructions for handling, storing, or using the components. In some embodiments, the kits contain one or more containers or vessels for stably shipping, storing, or using the reagents. In some embodiments, the kits further comprise components for administering the targeting components of the invention.

In some embodiments, the kits contain a detection component and/or software for facilitating detection and analysis of the imaging component.

In some embodiments, the present invention provides a system comprising two or more of: a first labeled targeting component (e.g., fluorescently labeled fibrinogen); a second labeled targeting component (e.g., radioactively labeled fibrinogen); a detection device (e.g., MRI, PET, or CT scanners, Geiger counters, light-emitting devices, etc.), software for use in imaging and analysis of data, surgical devices, components for creating or maintaining a sterile work area, buffers, control reagents, pharmaceutical agents, and instructions.

The present invention also provides reactions mixtures that contain one or more targeting components of the present invention. Reaction mixture may include cells, tissues, clots, and the like.

The compositions of the present invention may be used in a variety of surgical and research methods. In some embodiments, two or more compounds (e.g., an optically labeled fibrinogen and a radioactively labeled fibrinogen) are injected intravenously into a subject simultaneously or in series and the subject is placed in a nuclear medicine scanner to find a bleeding source. If surgery is required, a hand-held or other device is used to light up the bleeding site during the operation.

The methods of the present invention may be used for detection of bleeding in any part of the body, including, but not limited to: head, neck, torso, extremities, trachea, lungs, mediastinum, pleural space, esophagus, stomach, small intestine, large intestine, liver, spleen, kidneys, pancreas, gallbladder, retroperitoneum, pelvis, rectum, and anus.

Exemplary methods are described below.

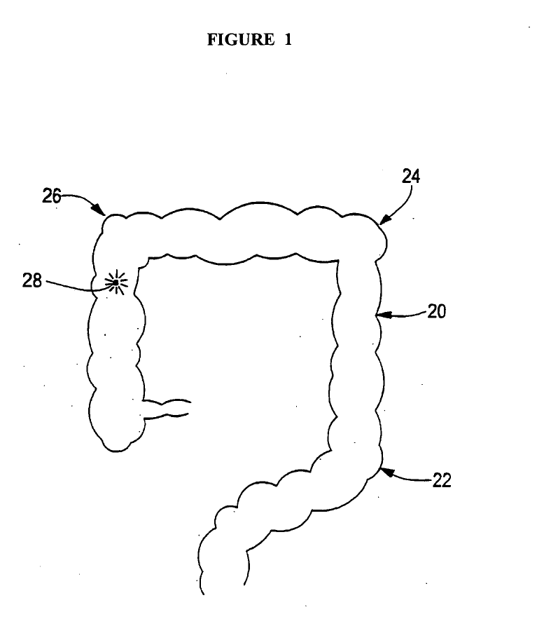

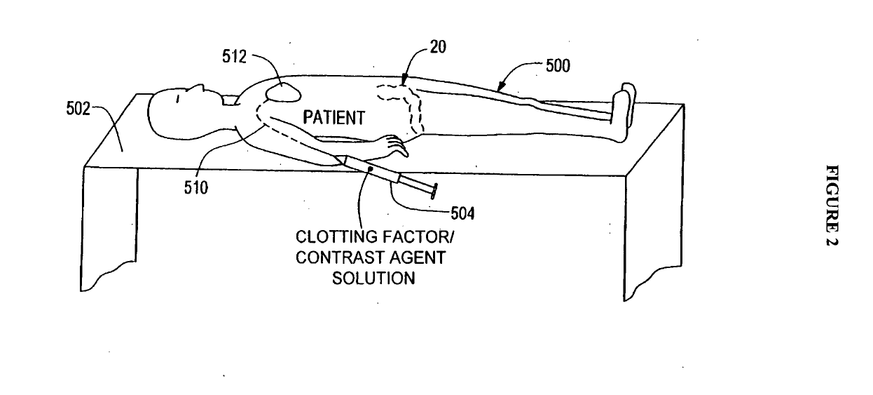

FIG. 1 shows a human colon 20 including a series of characteristic bends 22, 24, and 26 with a bleeding site 28 located past the bend 24. Bleeding in this location would be typical for diverticulosis. Such a bleeding site would be difficult to locate using endoscopy because this site is relatively inaccessible to endocscopes. Similarly, trying to identify such a bleeding site via angiography or a nuclear medicine scan might also fail.

Reference is now made to the coagulation cascade models that have been determined which govern the clotting of a bleeding site such as site 28 of FIG. 1. The most important mechanism that the body employs to stop bleeding is the formation of a clot. Clots are composed of platelets and a number of specialized proteins. At the time of bleeding they collect at the site of a hemorrhage and a clot begins to form. Clotting is a dynamic process that involves initial platelet and protein deposition followed by a continuing deposition process and remodeling.

Bleeding occurs at the site of damage to the layer of cells that line blood vessels. This results in a hole in the vessel that allows egress. The body attempts to repair this hole in the following manner. Platelets and proteins in the blood stream are attracted to the damage by proteins produced by damaged cells and substances in the vessel wall which are exposed by the damage.

These proteins and platelets begin the formation of a plug which will eventually grow to cover and repair the defect. Initially this plug is composed of proteins and platelets. This plug is temporary, and after the bleeding is successfully halted, a complex series of molecular events occurs which greatly increases the strength and durability of the plug and allows the cells underneath the clot to heal and reestablish normal functioning.

Historically, the molecules involved in clotting were divided into two distinct pathways, the intrinsic and extrinsic pathways. Recent work has shown there is considerable overlap between the two pathways and it is more useful to think of the pathways together. The precise details of the clotting cascade are not known, but the major aspects of is clotting are as follows. Damage to a blood vessel exposes tissue factor and other factors that cause platelets to adhere at the site. After a series of molecular events, activated factor VIIa and phospholipid convert factor IX to IXa and X to Xa. These molecules contribute to the production of thrombin (Factor II) from its precursor. Thrombin then converts fibrin to its active form. Fibrin is one of the principal proteins making up the clot. This process of clot formation is a dynamic one, in which weaker areas of the clot may rupture, necessitating repeat of the process in a localized area. In this way, new molecules and platelets are constantly being recruited to the site of bleeding.

At the final stage, activation of Factor XIII helps to crosslink fibrin, which stabilizes the clot. Over time the fibrin molecules will link with each other in a dense mesh to form a durable clot. This clot will typically remain for a few days to weeks, depending on the size of the initial hemorrhage.

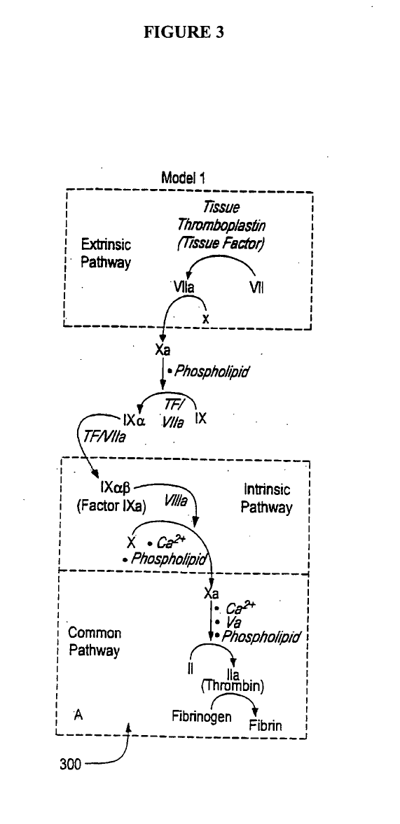

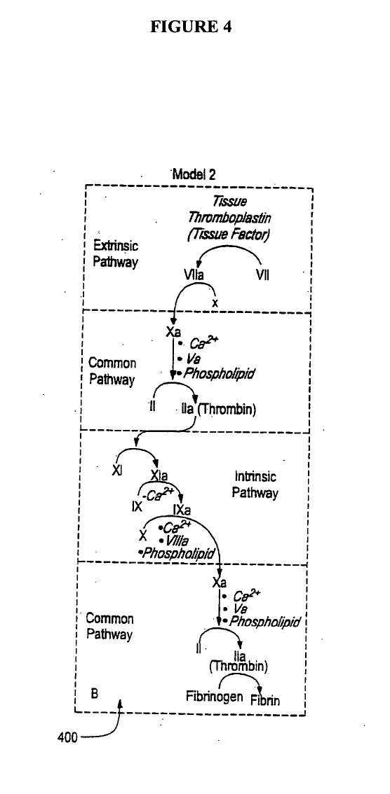

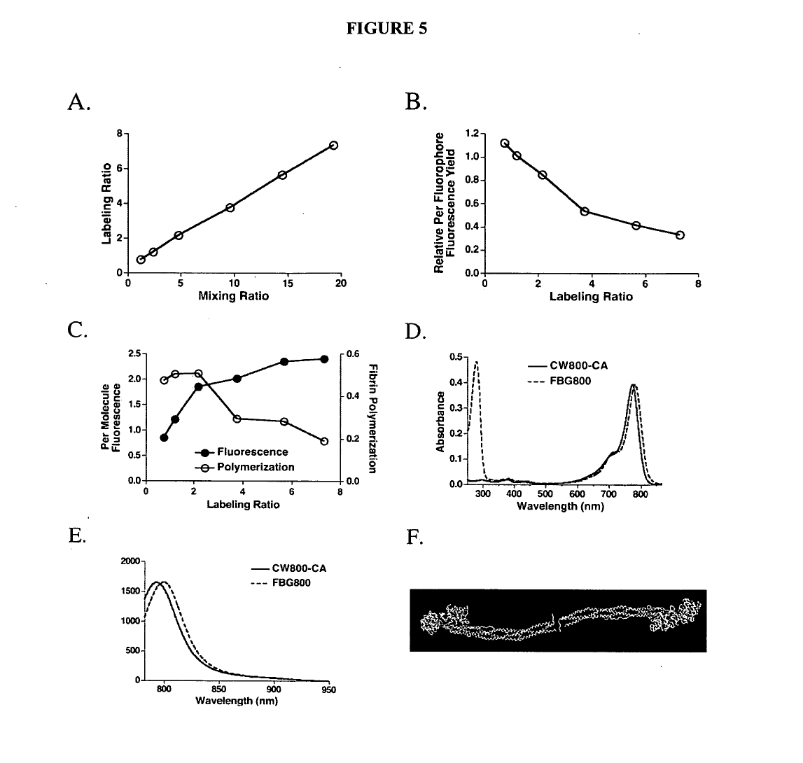

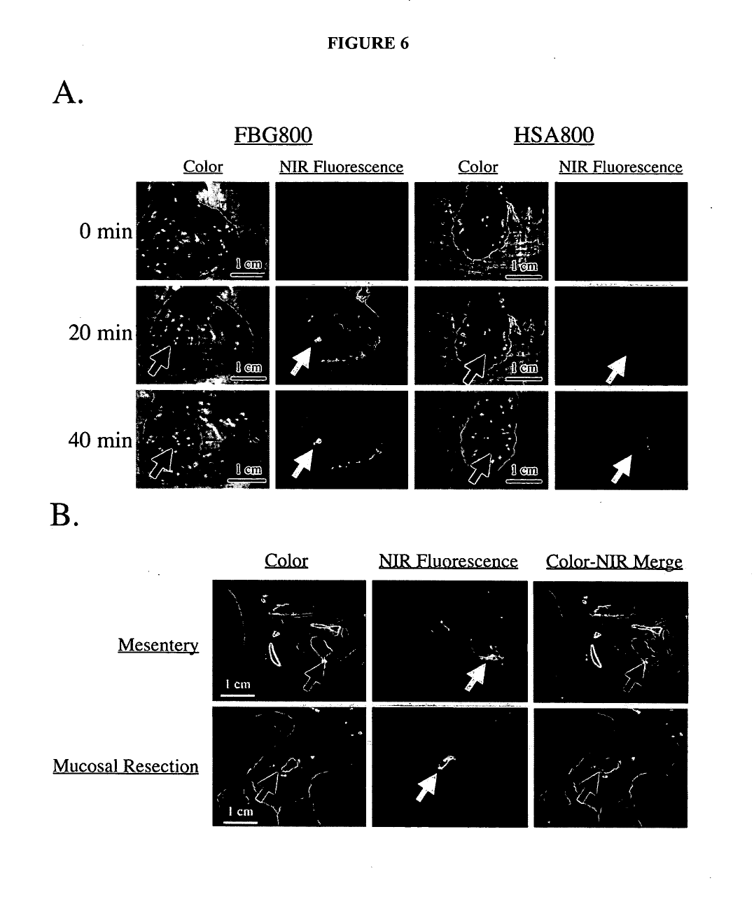

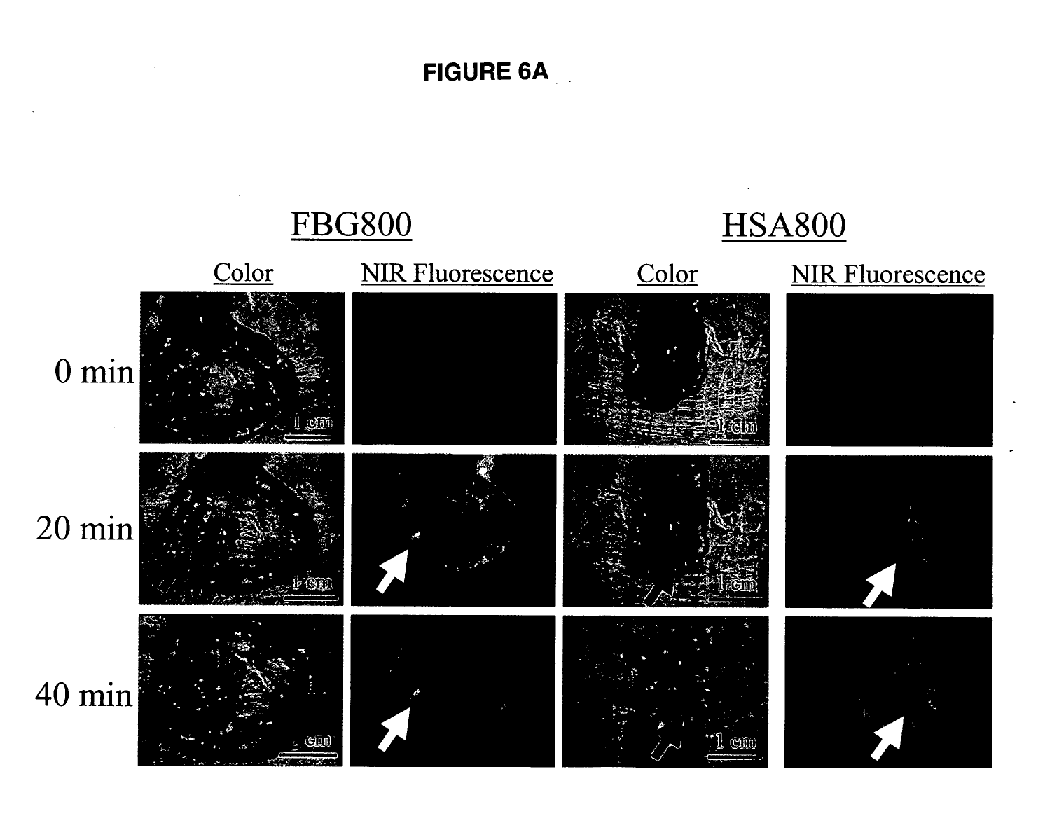

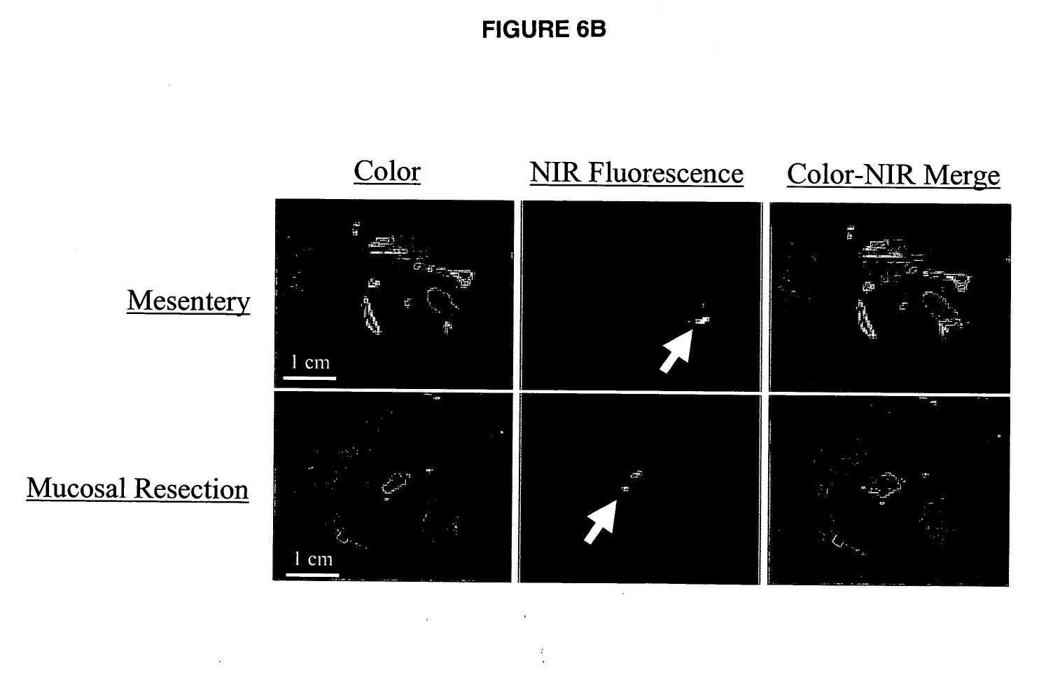

The various factors recruited by the site from the blood stream to enable clotting are shown in FIGS. 3 and 4. The factors in each of the models 300 (FIG. 3) and 400 (FIG. 4) are combined with other elements in the bloodstream such as calcium and phospholipids to eventually form the final clotting products fibrinogen and fibrin. Even if full clotting does not occur, clotting material will be continuously deposited at the bleeding site. And because clotting material is virtually always present, the present invention utilizes the presence of such material as the basis for detecting the exact location of the bleeding site. In this connection, it is noted that, in general, most of the clotting factors will not be present in high concentration in portions of the body other than the actual bleeding site. Thus, by applying an appropriate imaging contrast agent to one or more of the clotting factors shown in the coagulation cascade models 300 and 400 of FIGS. 3 and 4, a product for detecting clotting can be produced.

The following clotting factor or factors, alone and in combination, can be utilized according to this invention: platelets, Factors I, Ia, II, IIa, V, Va, VII, VIIa, VIII, VIIIa, IX, IXa, X, Xa, XI, XIa, XII, XIIa, XIII, XIIIa, fibrinogen, fibrin, fibronectin, von Willebrand's Factor, vinculin, vitronectin, Factor VIIIa and/or b component peptides, ADP, serotonin, platelet factor 4, bethathromboglobulin, high-molecular-weight kininogen, kallikrein, prekallikrein and antithrombin III. Other factors involved in the clotting process can also be used. In general, the factor(s) used should have enough longevity in the clotting process, or should result in by-products that have enough longevity in the clotting process so that they are not reabsorbed into the system too quickly. In addition, the factor(s) used should not be deleterious to health when administered in a detectable concentration and should be present in clots at some time in sufficient concentrations to be detectable. In addition, the factor(s) used should remain sufficiently diluted in other parts of the body so that they do not trick the detector into giving a false reading. In other words, the factor(s) used should not have a substantial affinity for other organs or locations other than the clotting site. Also, the factor(s) used should exhibit sufficient build-up at the clotting site, in a reasonable short period of time, so that they are detectable over and background “noise” generated by remaining freely circulating labeled factors.

The imaging contrast agent may, for example, be one or more of the many known contrast agents used in MRI, CT, PET, fluorescence, or other imaging techniques. Suitable MRI contrast agents include paramagnetic metals such as titanium, vanadium, chromium, manganese, iron, cobalt, nickel, copper, europium, gadolinium and protactinium as disclosed in U.S. Pat. No. 4,615,879 to Runge et al, or a ferromagnetic or superparamagnetic iron oxide such as magnetite and gamma ferric oxide as disclosed in U.S. Pat. No. 4,827,945 to Groman et al. Suitable CT contrast agents include any x-ray opaque material such as iron, calcium, barium, iodine, as routinely used in radiographic imaging. Suitable PET agents include Fluorine 18. Ideal fluorescent substances emit, after suitable excitation, wavelengths to which body tissue and blood are transparent. This allows them to be detected by various imaging equipment. Fluorescent substances that emit in the infrared range have this property, and are therefore ideal imaging agents. Examples include indocyanine green, iodocyanine green, IRDye78, IRDye80, IRDye38, IRDye40, IRDye41, IRDye700, IRDye800, Cy7, IR-786, DRAQ5NO, quantum dots, and analogs thereof. Quantum dots in particular are semiconductor nanocrystals with size-dependent optical and electronic properties such that when illuminated with a primary energy source, they emit at a specific energy frequency. The list of fluorescent substances should not be taken to be complete. Any molecule that can be excited to emit light in the infrared or near-infrared could be similarly complexed to a protein or substance involved in clotting could similarly be used. The entire contents of Runge et al. and Groman et al., are incorporated herein by reference.

The method of complexing the contrast agent to the protein is dependent on both the agent and the clotting factor. This is generally done using existing techniques. For example, iodination of a protein is a well-known method. The complexed product is provided in an aqueous solution that can include other elements such as saline. More specifically, the complexed product can be provided in concentrations of approximately one milligram per milliliter.

Variants of the current invention include the use of proteins with significant sequence homology to the proteins involved in the clotting process, or formulations employing fragments or portions of the proteins.

With reference now to FIG. 2, a technique for administering the complexed imaging contrast agent and clotting factor solution of the present invention for locating an internal bleeding site is explained. The patient 500 is shown reclining on an examination or operating room table 502. Alternatively, the patient can be seated or even standing. A syringe 504 or other device for delivering the solution is applied to the patient's circulatory system shown here as a series of dotted lines 510 interconnected to the heart 512. Typically, a vein in the arm is used. The syringe, an IV bag or some other device carries the solution. The total volume of solution injected can be approximately 10-100 milliliters administered over a time period of approximately a few minutes. The solution enters the bloodstream through the circulatory system 510 and eventually migrates throughout the body until it finds its way to the bleeding site in the colon 20. Clotting is an ongoing process. The approximate time from administration of the solution to the build-up of a sufficiently detectable concentration is approximately a few minutes. At such time, a CT, MRI or PET machine, or a fluoroscope or other appropriate imaging apparatus is utilized to locate the site of the bleeding in the patient by detecting the contrast agent so as to determine a location of the bleeding site based upon a concentration of the contrast agent complexed to the clotting factor.

The foregoing provides a detailed description of a preferred embodiment. Various modifications and additions can be made without departing from the spirit and scope of the invention. In particular, the list of clotting factors set forth herein should not be taken as exhaustive. Additional proteins and other materials involved in various stages of the clotting process can be employed. Similarly, various additional contrast agents other than the contrast agents described herein may be complexed to the clotting factor without departing from the scope of the invention. In addition, the times and dosages described herein can be varied. For example, multiple administrations of different clotting factors and/or different types of clotting factors in each administration can be employed. Accordingly, the foregoing description is meant to be taken only by way of example and not to otherwise limit the scope of the present invention as defined in the appended claims.

### EXAMPLE

This example provides an illustrative embodiment of the concepts of the invention described above. The present invention is not limited to the specific embodiment described below. This embodiment provides a radiolabeled ¹²⁵I-FBG and an 800-nm fluorescent FBG that, in conjunction with the appropriate imaging system, can be used to detect enzymatically active clot even after cessation of bleeding. The fluorescence of FBG800 has been optimized for 800 nm, a region of the spectrum with low autofluorescence and scatter, and fluorophore substitution has been titrated carefully to simultaneously maximize photon yield and biopotency. ¹²⁵I-FBG and FBG800 are complementary in their use, creating a system for the diagnosis and treatment of obscure GI bleeding.

### SUMMARY

This example tested two novel fibrinogen (FBG)-based contrast agents for their ability to localize gastrointestinal (GI) hemorrhage after bleeding stopped. ¹²⁵I-FBG permits gamma ray-based preoperative or intraoperative scanning, and near-infrared (NIR) fluorescent FBG (FBG800) permits real-time intraoperative visualization of active clot.

Bovine FBG was radiolabeled with ¹²⁵I or conjugated to the NIR fluorophore CW800. Sites of bleeding were created by gastrotomy, mucosal resection of the stomach, or laceration of a mesenteric vessel; then 1.7 mg/kg FBG800 or 15 μCi/kg ¹²⁵I-FBG was injected intravenously into mice, rabbits, or pigs 30 minutes before or after injury. Sites of active clot were quantified by using gamma counting and were also imaged by using invisible NIR light intraoperatively, for up to three hours postinjection.

After an injection of either ¹²⁵I-FBG or FBG800, sites of prior bleeding could be identified in the absence of active bleeding. Blood clearance was such that a signal-to-background ratio of 2.0 or greater could be achieved within 20 minutes after injection. A similarly labeled human serum albumin did not accumulate at any site, with an SBR of 1.0 or less.

Both radiolabeled (preoperative gamma scanning) and NIR fluorescent (intraoperative real-time imaging) FBG can be used in experimental situations to identify the location of prior bleeding in the absence of active bleeding. Taken together, these contrast agents create a system for the identification and control of obscure GI bleeding.

### BACKGROUND

Gastrointestinal (GI) bleeding is a common disorder and a significant source of morbidity and mortality. Determining the site of bleeding is often difficult, and the inability to identify the source of bleeding results in prolonged hospitalization and compromises treatment. Colonoscopy, endoscopy, nuclear scans, and angiography often fail to identify a source of bleeding because the bleeding from a particular site, although severe, may be intermittent. Radiolabeled red blood cell scans require active bleeding at a rate of 0.1 to 0.4 mL/min,¹ whereas angiography requires bleeding at a rate in excess of 0.5 mL/min.² If bleeding recurs after a negative test, a total colectomy, a major and morbid procedure, may be required.^{3,4} Preoperative or intraoperative localization would result in a much smaller and less-morbid operation.

At all sites of bleeding, injury to the blood vessel wall triggers blood coagulation.^{5,6} Platelets adhere to endothelial proteins in the injury bed, followed by aggregation and fibrin formation. Fibrinogen (FBG), a circulating glycoprotein, plays an important role in the blood-clotting process.^{7,8,9} Radiolabeled FBG has been used for the imaging of vascular thrombus. However, the use of FBG for the detection of bleeding sites has not been reported.^{10,11}

The present invention provides that attaching labels to FBG that allows it to be detected either via radioactive gamma ray emission or light-based fluorescence emission to permit detection of bleeding sites long after obvious bleeding has stopped because a fresh clot remains enzymatically active with respect to the clotting cascade. Intraoperative near-infrared (NIR) fluorescence imaging systems that permit anatomy (using color video) and function (using NIR fluorescence) to be acquired simultaneously and in real-time have been developed.¹² Because of high photon penetration and low autofluorescence in the 700- to 900-nm wavelength range, invisible NIR light is favorable for intraoperative imaging^{13,14,15} and, most importantly, does not alter the look of the surgical field. In this report, we describe the detection of active clot, in the absence of active bleeding using both radioactive and optical contrast agents, in 3 different animal model systems.

Methods

Reagents

The N-hydroxysuccinimide (NHS) ester (CW800-NHS) and carboxylic acid (CW800-CA) forms of IRDye™ 800CW NIR dye were provided as dry powders from LI-COR (Lincoln, Nebr.). They were resuspended at 30 mmol/L in dimethylsulfoxide (Sigma, St Louis, Mo.) under reduced light conditions and stored at −80° C. Bovine FBG was purchased from MP Biomedicals (Irvine, Calif.), dissolved at 20 mg/mL in phosphate-buffered saline (PBS, pH 7.8), and stored at −20° C. Human thrombin (2000 NIH units/mg protein) was purchased from Sigma and dissolved at 50 U/mL and stored at −80° C. Radiolabeled fibrinogen (¹²⁵I-FBG) and human serum albumin (¹²⁵I-HSA) were purchased from Amersham Biosciences (Piscataway, N.J.).

Conjugation Reactions for NIR Fluorescent FBG

All steps were performed under reduced light conditions. Reactions contained 15 mg/mL FBG and various molar ratios of CW800-NHS in PBS with reaction volumes ranging from 100 μL (analytical) to 5 mL (preparative). Conjugation was initiated by adding CW800-NHS, and constant agitation (without frothing) was continued for 2 hours at room temperature. Quenching of unreactive NHS esters was not necessary given the purification system used (see below).

Gel Filtration Chromatography

The gel filtration chromatography system consisted of an ÄKTA prime pump with fraction collector (Amersham Biosciences, Piscataway, N.J.) and Econo-Pac P6 chromatographic cartridge with a cutoff of 6000 Da (Bio-Rad, Hercules, Calif.). Gel filtration and online absorbance and fluorescence spectrometry was performed as described in detail previously.¹⁶ After conjugation, the sample was loaded into the injector and run at a flow rate of 1 mL/min with the use of PBS as mobile phase. Full-spectrum absorbance and fluorescence data were recorded every 10 seconds. Desired products were collected by the fraction collector, pooled, and stored at 4° C. in the dark without preservatives until use. Average yield was 85% or greater. The labeling ratio and the concentration of the conjugated protein (FBG800) were estimated by using the extinction coefficients of FBG (ε₂₈₀ₙₘ=330,000 M⁻¹cm⁻¹) and CW800-CA (ε₇₇₇ₙₘ=242,000 M⁻¹cm⁻¹) in PBS, with correction for the 6.5% of measured absorbance at 280 nm attributable to CW800-CA:
Labeling Ratio=(Abs₇₇₇ₙₘ/_{□777nm})/((Abs₂₈₀ₙₘ−0.065*Abs₇₇₇ₙₘ)/□₂₈₀ₙₘ)

Electrospray Time-of-Flight Mass Spectroscopic Site Mapping

Two hundred thirty microliters of 8 mol/L urea/400 mmol/L ammonium bicarbonate solution was added to 1 mg FBG or FBG800 in 20 μL of PBS. Five microliters of 45 mmol/L dithiothreitol was added and incubated for 15 minutes at 50° C. Five microliters of 100 mmol/L iodoacetamide was added and incubated for 15 minutes at room temperature. The mixture was diluted with 750 μL of buffer (80 mg ammonium bicarbonate and 6 mg CaCl₂ in 10 mL H₂O), 20 μg of TPCK-trypsin (L-1-tosylamide-2-phenylethyl ketone-treated, Sigma) in water was added, and the solution was incubated for 24 hours at 37° C. Twenty microliters of this peptide digest was used for analysis on a Waters (Milford, Mass.) LCT electrospray time-of-flight liquid chromatography/mass spectrometry equipped with dual wavelength absorbance detector (Waters), multiwavelength fluorescence detector (Waters), a Sedex Model 75 evaporative light scatter detector (ELSD; Richards Scientific, Novato, Calif.), and a lock-spray. The absorbance detector was set to 254 and 700 nm (the maximum permitted wavelength), and the fluorescence detector was set to excite at 770 nm and detect emission at 800 nm. Leucine enkephalin (0.5 ng/μL) was used as a mass reference. Buffer A was 10 mmol/L triethylammonium acetate, pH 7 (Glen Research, Sterling, Va.), and buffer B was acetonitrile. Peptides were resolved on a 2.1×150 mm Symmetry C₁₈ column (Waters) at a flow rate of 0.3 mL/min by using a gradient of 5% to 40% B over 35 minutes. Mass was measured in ES+ mode. Data were analyzed with MassLynx (Waters) software, and expected peptide masses were calculated from the mass obtained from Peptidemass (Internet site at au.expasy.org, then, tools, then, peptide-mass.html) with the addition of the mass of CW800-CA (1003.24) and subtraction of the mass of H₂O (18.02). Three-dimensional protein structure was visualized on a Macintosh iMac computer running RasMac Molecular Graphics version 2.6-ucb1.0 (University of California, Berkeley, Calif.).

Thrombin-Catalyzed Fibrin Polymerization

Polymerization was followed by turbidity at 350 nm with the use of an HR2000 fiber optic spectrometer connected to a DH-2000-BAL light source and CUV-ALL-UV 4-way cuvette holder (Ocean Optics, Dunedin, Fla.). FBG800 (497 μL of 0.5 mg/mL) of various labeling ratios in PBS was mixed with human thrombin (3 μL of 50 U/mL) and placed into the cuvette immediately. Turbidity was monitored at room temperature for 30 minutes. Maximum slope was obtained from the turbidity curves, and relative slope was calculated from the slope of FBG alone, which was set to 1.0.

Animal Models of Bleeding

Animals were housed in an AAALAC-certified facility and were studied under the supervision of an approved institutional protocol. CD-1 mice weighing 30 g were from Charles River Laboratories (Wilmington, Mass.). Adult New Zealand White rabbits weighing 3 kg were from Robinson Services (Mocksville, N.C.). Yorkshire pigs weighing 30 kg were from E. M. Parsons and Sons (Hadley, Mass.). All animals acclimated to the animal facility for at least 48 hours before experimentation and were euthanized after experimentation with the use of pentobarbital (mice) or rapid intravenous injection of Fatal-Plus (Vortech Pharmaceuticals, Dearborn, Mich.; rabbits and pigs).

Anesthesia was induced with an intraperitoneal injection of pentobarbital (40 mg/kg) to mice and an intramuscular injection of ketamine (35 mg/kg) and xylazine (5 mg/kg) to rabbits. Pig anesthesia was induced with 4.4 mg/kg intramuscular Telazol (Fort Dodge Labs, Fort Dodge, Iowa), and anesthesia was maintained through a 7-mm endotracheal tube with 1.5% isoflurane/98.5% O₂ at 5 L/min. After anesthesia, a laparotomy was made in the midline; the stomach and a loop of small intestine were externalized. A mesenteric vessel of the mice and pigs was lacerated and then permitted to clot, resulting in intramesenteric thrombus formation. For rabbits and pigs, a gastrotomy was made, and the posterior wall mucosa was pulled through. A 5-mm mucosectomy was performed with a scalpel. The injured area was allowed to clot and marked with a 4-0 silk suture for identification.

Contrast Agent Administration

1.7 mg/kg FBG800 protein (10 nmol/kg of fluorophore) was injected intravenously in mice (N=4) and pigs (N=3). 15 μCi/kg was injected intravenously in rabbits (N=3). This dose is approximately 100 ng and is much less than the total fibrinogen in the rabbit. Injections occurred 30 minutes before or 30 minutes after injury as described below. For controls, the same dose of CW800-labeled human serum albumin (HSA800, N=3)¹⁶ or radiolabeled albumin (¹²⁵I-HSA, N=3) was used.

Intraoperative Imaging System

The intraoperative NIR fluorescence imaging system optimized for animal surgery has been described previously in detail.¹² Briefly, it is composed of 2 wavelength-isolated excitation sources, one generating 0.5 mW/cm² of 400- to 700-nm “white” light and the other generating 5 mW/cm² of 725- to 775-nm light over a 15-cm diameter field of view. Simultaneous photon collection of color video and NIR fluorescence images is achieved with custom-designed optics that maintains separation of the white light and NIR fluorescence (>795 nm) channels. Spatial resolution at a field-of-view of 20×15 cm is 625 μm, and at a field-of-view of 4×3 cm is 125 μm. After computer-controlled (LabVIEW) camera acquisition via custom LabVIEW (National Instruments, Austin, Tex.) software, anatomic (white light) and functional (NIR fluorescence light) images could be displayed separately or merged. All images are refreshed up to 15 times per second. The entire apparatus is suspended on an articulated arm over the surgical field, thus permitting noninvasive and nonintrusive imaging.

Signal Intensity Measurements

After injection of FBG800, signal to background ratio (SBR) was measured by using as background the signal from an identically sized and shaped region of interest over adjacent mesenteric vessels or GI mucosa. After injection of ¹²⁵I-FBG, a standard Geiger counter was swept over the areas of injury to determine whether the site of injury could be recognized simply on the basis of meter readings. The entire stomach was then removed and opened. A portion of stomach away from both the gastrotomy site and the site area of mucosal injury was resected and used for background measurements. Next, the gastrotomy site was resected. Finally, the area of mucosal injury was recognized and resected. All tissues were weighed, placed in scintillation vials, and counted in a standard scintillation counter. Statistical comparisons were made with a Student t test.

Results

Synthesis of FBG800 and its Optical and Biologic Properties

After reaction with fluorophore, FBG800 was successfully separated from free NHS ester and free CW800-CA with a purity of >95% by gel filtration. The labeling ratio increased as the mixing ratio increased (FIG. 1, A). However, as the labeling ratio increased, the relative per-fluorophore fluorescence decreased (FIG. 1, B) and the per-molecule fluorescence plateaued (FIG. 1, C). Because of this phenomenon, it was concluded that maximal per-molecule fluorescence yield occurs at a labeling ratio of 6 to 8 (FIG. 1, C).

However, increasing fluorophore substitution resulted in marked changes in biopotency. Even at a labeling ratio of 1.0, FBG800 bioactivity was 50% of native FBG (FIG. 1, C). Above a labeling ratio of 2.0, FBG polymerization was inhibited significantly (FIG. 1, C). FBG at a labeling ratio of 2.0 was therefore used for all further studies. Peak absorption of CW800-CA and FBG800 was 775 nm and 785 nm, respectively (FIG. 1, D). Peak fluorescence emission of CW800-CA and FBG800 was 794 nm and 800 nm, respectively (FIG. 1, E), showing the characteristic bathochromic shift after conjugation.¹⁶

Identification of Fluorophore Location(s) on FBG800

To determine the conjugation site(s) of CW800, tryptic mass-spec fingerprinting was performed. The total ion chromatogram demonstrated that peptides from both FBG and FBG800 were well separated, with a small number of peaks of 700 nm absorbance (the highest available wavelength on our detector) and 800 nm fluorescence observed from FBG800, but not from FBG. By measuring the mass of the peptides with 700-nm absorbance and 800-nm fluorescence, 2 conjugation sites were obtained, at amino acids Lys399 and Lys185 of the Bβ chain. Fortunately, neither conjugation site (FIG. 1, F) is located in the fibrinopeptide A or B,⁹ which are released by thrombin cleavage and are not included in the fibrin clot.⁸

Intraoperative Thrombus Detection with ¹²⁵I-FBG

In some embodiments, radiolabeled FBG is used with a gamma camera to confirm occult GI bleeding and to partially localize the bleeding site. For proof of principle, the low-energy gamma emitter ¹²⁵I was used. In the first group, rabbits injected 30 minutes before gastrotomy were analyzed. Radioactivity was counted 3 hours after the injury, and the counts at the sites of injury were divided by the counts at the uninjured area to evaluate the fold increase over background. The ratio of the counts at the site of injury using ¹²⁵I-FBG as compared to background, uninjured mucosa was 4.8±1.0 (N=3). The ratio of the counts at the site of the gastrotomy as compared to the uninjured mucosa was 6.9±1.8 (N=3). Importantly, the gastrotomy site and the injury site were readily identified in situ by a standard handheld Geiger counter. To evaluate whether the increased counts were due to a nonspecific effect of protein accumulation at the sites, ¹²⁵I-HSA was used as a control. In these animals, the ratio of the counts at the site of mucosal injury, as compared to the uninjured mucosa, was 1.7±1.0 (N=3) and at the gastrotomy, as compared to the uninjured mucosa, was 1.0±0.7 (N=3). There was a statistically significant difference between the ratios in the injured tissue with fibrinogen as compared to the ¹²⁵I-HSA control (P<0.05).

In the second group, rabbits injected 30 minutes after gastrotomy with either ¹²⁵I-FBG or ¹²⁵I-HSA were analyzed. The ratio of the counts at the site of the injury using ¹²⁵I-FBG as compared to background, uninjured mucosa was 3.0±0.5 (N=3). The ratio of the counts at the site of the gastrotomy as compared to the uninjured mucosa was 2.4+1.0 (N=3). The differences were statistically significant (P<0.05). Again, the gastrotomy site and the injury site were readily identified by a standard handheld Geiger counter. To evaluate whether the increased counts were due to a nonspecific effect of protein accumulation at the sites, ¹²⁵I-HSA was used as a control. In these animals, the ratio of the counts at the site of mucosal injury, as compared to the uninjured mucosa, was 1.2±0.1 (N=3) and at the gastrotomy, as compared to the uninjured mucosa, was 1.0±0.4 (N=3). There was a statistically significant difference between the ratios when ¹²⁵I-FBG was used as compared with ¹²⁵I-HSA (P<0.05).

Real-Time Intraoperative Thrombus Detection with FBG800 Using Invisible NIR Fluorescent Light

In the mice (N=4), FBG800 was injected intravenously 30 minutes after mesenteric vessel injury, when no active bleeding was seen. As shown in FIG. 2, A, FBG800 rapidly accumulated at the site of thrombus formation, and within 20 minutes SBR was optimal. The control protein HSA800 showed no accumulation (FIG. 2, A, N=4). The SBR of the clot was ≈2.0, whereas that of the adjacent area remained <1.0. To evaluate the performance of FBG800 in animals approaching the size of humans, mesenteric vessel injury and mucosal resection of the stomach were performed in 30-kg Yorkshire pigs (N=3). 30 minutes after these injuries, when no active bleeding was present, FBG800 was injected intravenously. It was found to accumulate rapidly at the sites of clot formation, with optimal SBR achieved by 30 minutes (FIG. 2, B). Intravenous injection of HSA800 resulted in no accumulation at any site of injury.

Discussion

Current methods for detecting GI bleeding all require active bleeding to localize the source. High false-negative rates of these tests are due to the fact that GI bleeding is often intermittent. There is a tremendous need for a test that can detect the site even if bleeding is not ongoing at the time of the test. The experiments above describe two novel contrast agents that do not require active bleeding to find the site of clot, and thus, previous hemorrhage. Although the experiments focused on FBG-based contrast agents, other components of the clotting cascade may be used, or markers on the endothelium, could be used in a similar fashion, but with different intensities, uptake, and decay characteristics.

The experiments demonstrated in three different animal model systems, ¹²⁵I-FBG and FBG800 can be administered before, during, or after injury and still be used to identify the site of bleeding. Since the size of the FBG protein is rather large (330 kDa), the plasma half-life is contemplated to be several days,¹⁷ which is helpful to the surgeon because these agents can be given to patients at high risk for rebleeding, as many patients are, and thus identify the bleeding source if, and when, rebleeding occurs. Radiolabeled and/or NIR fluorophore-labeled FBG can be administered to patients at presentation, in combination with methods that are currently being used, to detect bleeding or to increase the sensitivity of conventional tests. Although ¹²⁵I-FBG was used in this study for proof of principle, ¹³¹I-FBG provides the capability for full-body gamma ray scanning of humans. In terms of clinical use, radiolabeled and NIR fluorescent FBG are complementary. Radiolabeled FBG does not require a laparotomy as does FBG800, and it provides full-body scanning; FBG800 needs to be used in an operative setting, but it does not expose patient and caregivers to radioactivity, and provides sensitive and high-resolution visualization of active clot.

A variety of clotting factors may be used. In the setting of massive bleeding and ongoing transfusion, irrigation of the bowel may be used to prevent or reduce problems associated with washing out of the radioactivity or fluorescence.

Optimization of the NIR fluorescent proteins such as FBG800 can be conducted, as desired for optimization of total fluorescent yield and biopotency. Although it is usually possible to achieve high substitution ratios (FIG. 1, B), internal filter effects and dye quenching limit total fluorescent yield (FIG. 1, C). Wide spacing of heptamethine indocyanines over the surface of a large protein can minimize, but not eliminate these effects.¹⁶ Fluorophore substitution can also alter the molecule's biopotency (FIG. 1, C).

### REFERENCES (HEREIN INCORPORATED BY REFERENCE IN THEIR ENTIRETIES)

1 R. Smith, D. J. Copely and F. H. Bolen, 99mTc RBC scintigraphy: correlation of gastrointestinal bleeding rates with scintigraphic findings, AJR Am J Roentgenol 148 (1987), pp. 869-874.
2 S. Baum, C. A. Athanasoulis, A. C. Waltman and E. J. Ring, Gastrointestinal hemorrhage, II: Angiographic diagnosis and control, Adv Surg 7 (1973), pp. 149-198.
3 R. Famer, W. Lichliter, J. Kuhn and T. Fisher, Total colectomy versus limited colonic resection for acute lower gastrointestinal bleeding, Am J Surg 178 (1999), pp. 587-591.
4 A. Schuetz and K. W. Jauch, Lower gastrointestinal bleeding: therapeutic strategies, surgical techniques and results, Langenbecks Arch Surg 386 (2001), pp. 17-25.
5 B. Dahlback, Blood coagulation, Lancet 355 (2000), pp. 1627-1632.
6 H. M. Spronk, J. W. Govers-Riemslag and H. ten Cate, The blood coagulation system as a molecular machine, Bioessays 25 (2003), pp. 1220-1228.
7 R. F. Doolittle, S. J. Everse and G. Spraggon, Human fibrinogen: anticipating a 3-dimensional structure, Faseb J 10 (1996), pp. 1464-1470.
8 C. Fuss, J. C. Palmaz and E. A. Sprague, Fibrinogen: structure, function, and surface interactions, J Vasc Interv Radiol 12 (2001), pp. 677-682.
9 S. Herrick, O. Blanc-Brude, A. Gray and G. Laurent, Fibrinogen, Int J Biochem Cell Biol 31 (1999), pp. 741-746.
10 S. T. Haines and H. I. Bussey, Diagnosis of deep vein thrombosis, Am J Health Syst Pharm 54 (1997), pp. 66-74.
11 L. C. Knight, Scintigraphic methods for detecting vascular thrombus, J Nucl Med 34 (1993), pp. 554-561.
12 A. M. De Grand and J. V. Frangioni, An operational near-infrared fluorescence imaging system prototype for large animal surgery, Technol Cancer Res Treat 2 (2003), pp. 553-562.
13 J. V. Frangioni, In vivo near-infrared fluorescence imaging, Curr Opin Chem Biol 7 (2003), pp. 626-634.
14 V. Ntziachristos, C. Bremer and R. Weissleder, Fluorescence imaging with near-infrared light: new technological advances that enable in vivo molecular imaging, Eur Radiol 13 (2003), pp. 195-208.
15 E. M. Sevick-Muraca, J. P. Houston and M. Gurfinkel, Fluorescence-enhanced, near infrared diagnostic imaging with contrast agents, Curr Opin Chem Biol 6 (2002), pp. 642-650.
16 S. Ohnishi, S. J. Lomnes, R. G. Laurence, A. Gogbashian, G. Mariani and J. V. Frangioni, Organic alternatives to quantum dots for intraoperative near-infrared fluorescent sentinel lymph node mapping, Mol Imaging 4 (2005), pp. 172-181.
17 C. P. Coyne, W. J. Hornof, A. B. Kelly, T. R. O'Brien and S. J. DeNardo, Extraction, radioiodination, and in vivo catabolism of equine fibrinogen, Am J Vet Res 46 (1985), pp. 2572-2577.
18 J. H. Brown, N. Volkmann, G. Jun, A. H. Henschen-Edman and C. Cohen, The crystal structure of modified bovine fibrinogen, Proc Natl Acad Sci USA 97 (2000), pp. 85-90.

It is claimed:

1. A kit comprising: a) an optically labeled targeting component configured to accumulate in a clot; and b) a radioactively labeled targeting component configured to accumulate in a clot.

2. The kit of claim 1, wherein said optically labeled targeting components is optically labeled fibrinogen.

3. The kit of claim 2, wherein said optically labeled fibrinogen comprises fluorescently labeled fibrinogen.

4. The kit of claim 1, wherein said radioactively labeled targeting component comprises radioactively labeled fibrinogen.

5. A kit comprising: a) an optically labeled targeting component configured to accumulate in a clot; b) an unlabeled targeting component and reagents for radioactively labeling said unlabeled targeting component.

6. The kit of claim 5, wherein said optically labeled targeting components is optically labeled fibrinogen.

7. The kit of claim 6, wherein said optically labeled fibrinogen comprises fluorescently labeled fibrinogen.

8. The kit of claim 5, wherein said unlabeled targeting component comprises fibrinogen.

9. A mixture comprising: a) an optically labeled targeting component configured to accumulate in a clot; and b) a radioactively labeled targeting component configured to accumulate in a clot.

10. The mixture of claim 9, wherein said optically labeled targeting components is optically labeled fibrinogen.

11. The mixture of claim 10, wherein said optically labeled fibrinogen comprises fluorescently labeled fibrinogen.

12. The mixture of claim 9, wherein said radioactively labeled targeting component comprises radioactively labeled fibrinogen.

13. The mixture of claim 9, wherein said first and second targeting components are provided in a pharmaceutically acceptable solution.

14. The mixture of claim 9, in pharmaceutical dosage form for administration to a subject at 1 to 100 microcuries/kg of body weight of said subject of radioactively labeled targeting component and 100 micrograms to 100 milligrams/kg of body weight of said subject of optically labeled targeting component.

15. A method for detecting bleeding in a subject, comprising administering an optically labeled targeting component configured to accumulate in a clot and a radioactively labeled targeting component configured to accumulate in a clot to a subject; and detecting localization of said optically labeled targeting component or said radioactively labeled targeting component in said subject.
